(19) United States
(12) Patent Application Publication
(10) Pub. No.: US 2021/0340315 A1
(43) Pub. Date: Nov. 4, 2021

(54) CASHEW NUT SHELL LIQUID DERIVATIVES AND METHODS FOR MAKING AND USING SAME

(71) Applicant: Cardolite Corporation, Bristol, PA (US)

(72) Inventors: Chetan Tambe, Bensalem, PA (US); Joseph Mauck, Newtown, PA (US); Pietro Campaner, Trieste (IT); Fernanda Tavares, Holland, PA (US); Anbazhagan Natesh, North Wales, PA (US); Timothy Stonis, Newtown, PA (US)

(73) Assignee: Cardolite Corporation, Bristol, PA (US)

(21) Appl. No.: 16/863,755

(22) Filed: Apr. 30, 2020

(51) Int. Cl. C08G 63/82; C07C 55/14; C07C 69/44; C07C 31/20; C07C 265/14; B01J 25/02; B01J 23/755; B01J 23/46; B01J 23/44; C08G 73/02

(52) U.S. Cl. CPC C08G 63/826; C07C 55/14; C07C 69/44; C07C 31/20; C08G 73/0213; B01J 25/02; B01J 23/755; B01J 23/462; B01J 23/44; C07C 265/14

(57) ABSTRACT

The present invention is directed to Cashew Nut Shell Liquid derivatives and methods for making and using same. The invention is also directed to Cashew Nut Shell Liquid based monomers and polymers for making antimicrobials, antioxidants, adhesives, coatings, corrosion retardants composites, cosmetics, detergents, soaps, de-icing products, elastomers, food, flavors, inks, lubricants, oil field chemicals, personal care products, polymers, structural polymers, engineered plastics, 3D printable polymers, techno-polymers, rubbers, sealants, solvents, surfactants and varnishes.

### BACKGROUND OF THE INVENTION

### Field of the Invention

Embodiments described herein generally relate to Cashew Nut Shell Liquid derivatives and methods for making and using same. More particularly, such embodiments relate to Cashew Nut Shell Liquid based monomers and polymers for making a wide variety of products.

### Description of the Related Art

Bio-based compounds have recently gained significant interest due to an increasing demand of sustainable alternatives to petroleum based raw materials. However, bio-based materials have showed some limitations relating to poor yields, by-products formation, limited selectivity, use of raw materials coming from the food chain, degradation, difficulties in selecting the optimal enzyme or microorganism, use of solvents, long reaction times, with an unavoidable impact on economic value, industrial scalability and sustainability.

Cashew Nut Shell Liquid (CNSL) is a well-known non-edible natural oil obtained as a by-product of the Anacardium occidentale nut. CNSL is one of the most widely used bio-based resource to provide useful chemicals for coatings, adhesives, sealants and elastomers (CASE) applications. Cardanol is an important chemical derived by decarboxylation of anacardic acid, which is the primary component of CNSL. However, there are some applications and sectors, where the use of cardanol is still quite limited due to lack of suitable building block derivatives. In fact, many of the cardanol derivatives reported in the literature are based on cardanol as mixture of isomers, with subsequent potential lack of reproducibility, UV instability, limited average type of functional groups and applicability in sectors where color and purity are key aspects (e.g. 1K polyurethanes, thermoplastics). Moreover, even if cardanol hydrogenation is known and the use of the resulting 3-pentadecyl-cyclohexanol is described in the literature, the number of examples of industrial applicability is still extremely limited.

Accordingly, there is still a need to provide increased functionality to cardanol derivatives. The present disclosure provides novel monomers, polymers and methods of making and using same. The present application satisfies these needs as well as others that are readily apparent to one skilled in the art.

### BRIEF SUMMARY OF THE INVENTION

Cashew Nut Shell Liquid derivatives and methods for making and using same are provided. In one embodiment, a compound of formula I is provided:

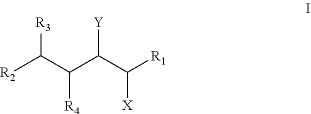

wherein:

X and Y are each independently H and C₁₅H₃₁;

R₁ and R₂ are each independently —COOH, —COOR₅, —CO-halogen, —CH₂OH, —CH₂OR₅, —COSR₅, —CONH₂, —CH₂NH₂, CH₂NHR₅, —CH₂N₃R₅, —NCO, —CH₂-halogen, —CH₂OCH₂(CHCH₂O), —CHO, —CN, —CONH—CO—R₅ and —CH₂O—CO—O—R₅; and

wherein R₅ is H, alkyl, alkenyl, alkoxy, cycloalkyl, aryl, heteroaryl, and —CH₂—N—(CH₂CH—R₆—O)₂—(CH₂CH—R₆—O)₀₋₁₀—H; and

wherein R₆ is H or alkyl;

R₃ and R₄ are each independently H, alkyl, alkoxy and cycloalkyl.

In other embodiment, a polymer of formula II is provided:

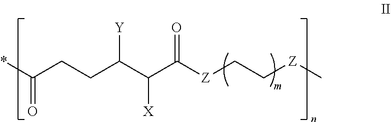

wherein:

X and Y are each independently H and C₁₅H₃₁;

Z is O, NH and S;

m is 1-20; and

n is 1-100.

In another embodiment, methods for preparing a compound of formula I and a polymer of formula II are provided.

In some embodiments, methods for using a compound of formula I and a polymer of formula II in antimicrobials, antioxidants, adhesives, coatings, corrosion retardants composites, cosmetics, detergents, soaps, de-icing products, elastomers, food, flavors, inks, lubricants, oil field chemicals, personal care products, polymers, structural polymers, engineered plastics, 3D printable polymers, techno-polymers, rubbers, sealants, solvents, surfactants, varnishes etc. are provided.

### BRIEF DESCRIPTION OF THE DRAWINGS

FIG. 1 illustrates an exemplary ¹H-NMR of fully hydrogenated cardanol (3-pentadecyl-cyclohexanol).

FIG. 2 illustrates an exemplary GC chromatogram of fully hydrogenated cardanol (3-pentadecyl-cyclohexanol).

FIG. 3 illustrates an exemplary ¹H-NMR of a mixture of isomers, 2-pentadecylhexanedioic acid and 3-pentadecylhexanedioic acid.

FIG. 4 illustrates an exemplary ¹³C-NMR of a mixture of isomers, 2-pentadecylhexanedioic acid and 3-pentadecylhexanedioic acid.

FIG. 5 illustrates an exemplary GPC chromatogram of a mixture of isomers, 2-pentadecylhexanedioic acid and 3-pentadecylhexanedioic acid.

FIG. 6 illustrates an exemplary GC chromatogram of a mixture of isomers, methyl 2-pentadecylhexanedioate and methyl 3-pentadecylhexanedioate.

### DETAILED DESCRIPTION OF THE INVENTION

Embodiments described herein generally relate to Cashew Nut Shell Liquid derivatives and methods for making and using same. More particularly, such embodiments relate to Cashew Nut Shell Liquid based monomers and polymers for making various products. Embodiments disclosed herein relate, in part, to the synthesis of a cardanol-based derivative through the full hydrogenation of any unsaturation present on cardanol-backbone, thus including all the double bonds present on cardanol's alkenyl C₁₅ side chain as well as cardanol's aromatic ring.

In some embodiments, the fully hydrogenated cardanol (3-pentadecyl-cyclohexanol) can be further transformed to a substituted alkyl adipic acid as mixture of isomers, namely 2-pentadecylhexanedioic acid and 3-pentadecylhexanedioic acid. These derivatives represent useful substrates for further chemical transformations, which include but are not limited to, for example, esterification, reduction to alcohols or conversion of carboxylic groups to isocyanate groups by Curtius rearrangement.

In other embodiments, these monomers can be used as versatile polymer building blocks, which include but are not limited to, for example, for the synthesis of polyester diols and polyols, polyethers, polyamides, epoxies, acrylates, alkyds, that can be used in 1K and 2K adhesives, elastomers, coatings, epoxy formulations, polyurethanes, thermoplastics, and the like.

As used herein, the term “alkyl” whether used alone or as part of another group, refers to a substituted or unsubstituted aliphatic hydrocarbon chain and includes, but is not limited to, straight and branched chains containing from 1 to 20 carbon atoms, preferably from 2 to 20, from 1 to 10, from 2 to 10, from 1 to 8, from 2 to 8, from 1 to 6, from 2 to 6, from 1 to 4, from 2 to 4, from 1 to 3 carbon atoms, unless explicitly specified otherwise. Illustrative alkyl groups can include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, 4,4-dimethylpentyl, octyl, 2,2,4-trimethylpentyl, nonyl, decyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

As used herein, the term “alkenyl” whether used alone or as part of another group, refers to a substituted or unsubstituted aliphatic hydrocarbon chain and includes, but is not limited to, straight and branched chains having 2 to 8 carbon atoms and containing at least one carbon-carbon double bond.

As used herein, the term “alkynyl” whether used alone or as part of another group, refers to a substituted or unsubstituted aliphatic hydrocarbon chain and includes, but is not limited to, straight and branched chains having 1 to 6 carbon atoms and containing at least one carbon-carbon triple bond.

As used herein, the term “alkoxy” whether used alone or as part of another group, refers to alkyl-O— wherein alkyl is hereinbefore defined.

As used herein, the term “cycloalkyl” whether used alone or as part of another group, refers to a monocyclic, bicyclic, tricyclic, fused, bridged or spiro monovalent saturated hydrocarbon moiety, wherein the carbon atoms are located inside or outside of the ring system. Any suitable ring position of the cycloalkyl moiety may be covalently linked to the defined chemical structures. Illustrative cycloalkyl groups can include, but are not limited to, cyclopropyl, cyclopropylmethyl, cyclobutyl, cyclopentyl, cyclohexyl, cyclohexylmethyl, cyclohexylethyl, cycloheptyl, norbornyl, adamantly, spiro[4,5]decanyl, and homologs, isomers and the alike.

As used herein, the term “aryl” whether used alone or as part of another group, refers to an aromatic carbocyclic ring system having 6 to 14 carbon atoms, preferably 5 to 10 carbon atoms, optionally substituted with 1 to 3 substituents independently selected from halogen, nitro cyano, hydroxy, alkyl, alkenyl, alkoxy, cycloalkyl, amino, alkylamino, dialkylamino, carboxy, alkoxycarbonyl, haloalkyl, and phenyl.

As used herein, the term “phenyl” whether used alone or as part of another group, refers to a substituted or unsubstituted phenyl group.

As used herein, the term “heteroaryl” whether used alone or as part of another group, refers to a 5 to 10 membered aryl heterocyclic ring, which contains from 1 to 4 heteroatoms selected from the group consisting of O, N and S atoms in the ring and may be fused with a carbocyclic or heterocyclic ring at any possible position.

As used herein, the term “heterocycloalkyl” whether used alone or as part of another group, refers to a 5 to 7 membered saturated ring containing carbon atoms and from 1 to 2 heteroatoms selected from the group consisting of O, N and S atoms.

As used herein, the term “halogen or halo” refers to fluoro, chloro, bromo or iodo.

As used herein, the term “haloalkyl” whether used alone or as part of another group, refers to an alkyl as hereinbefore defined, independently substituted with 1 to 3, F, Cl, Br or I.

As used herein, the term “about” refers that the numerical value is approximate and small variations would not significantly affect the practice of the disclosed embodiments. Where a numerical limitation is used, unless indicated otherwise by the context, “about” means the numerical value can vary by +10% and remain within the scope of the disclosed embodiments. Additionally, in phrase “about X to Y,” is the same as “about X to about Y,” that is the term “about” modifies both “X” and “Y.”

As used herein, the term “compound” refers to salts, solvates, complexes, isomers, stereoisomers, diastereoisomers, tautomers, and isotopes of the compound or any combination thereof.

As used herein, the term “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are used in their inclusive, open-ended, and non-limiting sense.

Cashew Nut Shell Liquid (CNSL) is a well-known non-edible natural oil obtained as a by-product of the Anacardium occidentale nut. CNSL is a non-food chain industrial oil found in the honeycomb structure of the cashew (Anacardium occidentale) nutshell, typically considered a by-product of the cashew nut industry. CNSL consists of a mixture of different chemical moieties (anacardic acid, cardanol, 2-methyl-cardol, cardol), all of them characterized by the presence of a C₁₅ side chain in the meta-position of the aromatic ring. This side chain contains a number of unsaturation from 0 to 3, with an average number of 2 double bonds. The main product isolated by vacuum distillation of CNSL under proper conditions is cardanol, which is an important chemical derived by decarboxylation of anacardic acid, as the primary component of CNSL. Cardanol is essentially, a meta-substituted phenol ring with mono-, di-, tri-unsaturated and saturated long 15-carbon chain, as shown below:

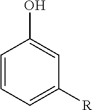

where R can be, for example:

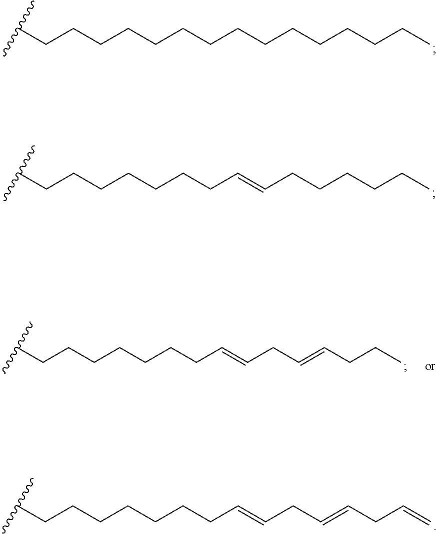 [SMILES: *CCCCCCCCCCCCCCC ; *CCCCCCC/C=C/CCCCCC ; *CCCCCCC/C=C/C/C=C/CCC ; *CCCCCCC/C=C/C/C=C/CC=C]

The peculiar cardanol structure, with an aromatic ring that provides excellent rigidity and thermal stability. It contains 15 carbon unsaturated aliphatic side chain at the meta position, imparting outstanding hydrophobicity. This aliphatic side chain may have either one, two or three carbon-carbon double bonds. This unsaturation can be used to derivatize useful chemicals from cardanol, which can be used in coatings, adhesives, antioxidants, elastomers, food, flavors, lubricants, polymers, rubbers, sealants etc. applications.

In some embodiments, the cardanol has a purity from about 80% to about 99.5% and preferably from about 95% to about 99.5%. Cardanol was treated under reductive conditions in order to hydrogenate all the double bonds on the C₁₅ side chain as well as on the aromatic ring yielding 3-pentadecyl-cyclohexanol in high purity and yield. Hydrogenation was carried on at a pressure from about 5 Bar to about 50 Bar and preferably from about 15 Bar to about 25 Bar and a temperature at about 30-200° C. and preferably at about 160-180° C. in presence of a catalyst. Illustrative catalysts can include, but are not limited to, Pd/C, Pd(OH)₂, Pd/Al₂O₃, Pd/NaY, Ru-PVP NPs, Ru/C, RuCl₃, Ni and Ni Raney or any combination thereof. The dosage level of the catalyst is about 1-10% w/w and preferably about 3-5% w/w, with or without the presence of a Lewis acid at about 5-20 mol % and preferably 8-12 mol %, without any limitations thereof. Illustrative solvents can include, but are not limited to, methanol, ethanol, isopropanol, n-butanol, hexane, cyclohexane, cyclopentane, N-methylpyrrolidone, N,N-dimethylformamide, dimethylsulfoxide, methylene chloride, chloroform, carbon tetrachloride, tetrahydrofuran, water or any combination thereof. The chemical structure of fully hydrogenated cardanol is shown below:

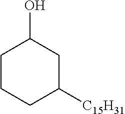

Even if cyclohexane (Van de Vyver, S.; Roman-Leshkov, Y. Catal. Sci. Technol. 2013 3(6), 1465-1479) and cyclohexene (Reed, S. M.; Hutchison, J. E. J. Chem. Ed. 2000, 77, 12, 1627-1629; Kazuhiko, S.; Masao, A.; Ryoji, N. Science, 1998, 281 (5383), 1646-1647; Vafaeezadeh, M.; Mahmoodi Hashemi, M. Chemical Engineering Journal 2013, 221, 254-257; Deng, Y.; Ma, Z.; Wang, K.; Chen, J. Green Chemistry, 1999, 275-276; Bailey, P. S. Ind. Eng. Chem. Res., 1958, 50, 7, 993-996), can be used to make adipic acid by the use of hydrogen peroxide, molecular oxygen or ozone, cyclohexanol and cyclohexanone are still the typical substrates, widely used at industrial level and described in the literature (Hermans, I.; Jacobs, P. A.; Peeters, J. Chem.-Eur. J., 2006, 12, 4229-4240).

Several approaches are reported for the conversion of cyclohexanol and cyclohexanone to adipic acid, as for example, their treatment in liquid phase under oxidative conditions at relatively high pressure (Drossbach, O. U.S. Pat. No. 2,285,914), or by the aim of using Oxone or ozone (d'Alessandro, N.; Liberatore, L.; Tonucci, L.; Morvillo, A.; Bressan, M. New J. Chem., 2001, 25, 1319-1324; Rokhum, L.; Bez, G. Synthetic Communications, 2011, 41, 548-552; Encinar, J. M.; Beltran, F. J.; Frades, J. M. Ind. Eng. Chem. Res., 1991, 30, 4, 617-623) or oxidizing agents like sodium nitrite in presence of tri-fluoro-acetic acid (Matsumura, Y.; Yamamoto, Y.; Moriyama, N.; Furukubo, S.; Iwasaki, F.; Onomura, O. Tetrahedron Letters, 2004, 45, 8221-8224), nitric acid and hydrogen peroxide are still the most widely used reagents currently used at industrial level (Chavan, S. A.; Srinivas, D.; Ratnasamy, P. Journal of Catalysis, 2002, 212, 39-45; Usui, Y.; Sato, K. Green Chemistry, 2003, 5, 373-375; Zhang, S.; Jiang, H.; Gong, H.; Sun, Z. Petroleum Sci. Technol., 2003, 21:1-2, 275-282; van Asselt, W. J.; van Krevelen, D. W. Recueil des Travaux Chimiques des Pays-Bas, 1963, 82, 1, 51-67; Bende, F.; Vollinger, H.; Pohl, K. U.S. Pat. No. 3,476,804; Castellan, A.; Bart, J. C. J.; Cavallaro, S. Catalysis Today, 1991, 9, 301-322; Van Asselt, W. J.; Van Krevelen, D. W. Chem. Eng. Sci., 1963, 18, 471-483; Castellan, A.; Bart, J. C. J.; Cavallaro Catalysis Today, 1991, 9, 285-299; Lindsay Smith, J. R.; Richards, D. I.; Thomas, C. B.; Whittaker, M. J Chem. Soc., Perkin Trans. 2, 1985, 1677-1682).

In some other embodiments, the fully hydrogenated cardanol (3-pentadecyl-cyclohexanol) was oxidized to a substituted alkyl adipic acid as mixture of isomers, namely 2-pentadecylhexanedioic acid and 3-pentadecylhexanedioic acid as shown below:

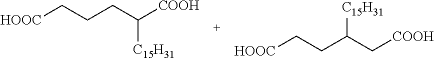

3-Pentadecyl-cyclohexanol was treated with an excess of nitric acid (with a concentration between 50% and 90% and preferably 65-70%). The number of nitric acid moles per each mole of 3-pentadecyl-cyclohexanol was between 4 and 20 and more preferable between 6 and 12 and even more preferably between 6 and 7. Ammonium vanadate and copper nitrate were used as catalysts, at a weight load level at about 0.05% and 0.4% (and preferably at about 0.1-0.3%) and at about 0.0% and 0.6% (and preferably at about 0.1-0.3%), respectively. Reaction was carried on at about 50° C. and 120° C., preferably at about 60° C. and 110° C. and more preferably at about 70° C. and 100° C. Given the peculiar structure of 3-pentadecyl-cyclohexanol and, in particular, the presence of the hydrophobic C₁₅ side chain, an organic acid like acetic acid can be optionally used in the synthesis to improve solubility and compatibility amongst all the reagents. The resulting alkyl-substituted adipic acid derivatives, 2-pentadecylhexanedioic acid and 3-pentadecylhexanedioic acid can be eventually further purified by recrystallization, if needed, or used as such.

Among the others, one major advantage of the present invention includes using 3-pentadecyl-cyclohexanol as the starting substrate for the synthesis of cardanol-derived adipic acid-like structures (2-pentadecylhexanedioic acid and 3-pentadecylhexanedioic acid). In fact, it can be easily recovered in high purity and yields.

Without being bound to any particular theory, one advantage of the methods and compounds described herein is the possibility to generate novel monomers with different types of functionalities that can be further reacted with other raw materials. For example, 2-pentadecylhexanedioic acid and 3-pentadecylhexanedioic acid can be used as starting materials for synthesizing novel monomers, which include, but not limited to, diesters, diols, and diisocyanates.

Accordingly, in some embodiments, a compound of formula I is provided:

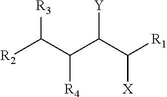

wherein:

X and Y are each independently H and C₁₅H₃₁;

R₁ and R₂ are each independently —COOH, —COOR₅, —CO-halogen, —CH₂OH, —CH₂OR₅, —COSR₅, —CONH₂, —CH₂NH₂, CH₂NHR₅, —CH₂N₃R₅, —NCO, —CH₂-halogen, —CH₂OCH₂(CHCH₂O), —CHO, —CN, —CONH—CO—R₅ and —CH₂O—CO—O—R₅; and

wherein R₅ is H, alkyl, alkenyl, alkoxy, cycloalkyl, aryl, heteroaryl, and —CH₂—N—(CH₂CH—R₆—O)₂—(CH₂CH—R₆—O)₀₋₁₀—H; and

wherein R₆ is H or alkyl;

R₃ and R₄ are each independently H, alkyl, alkoxy and cycloalkyl.

In some embodiments, the compound of formula I is provided as a mixture of diacids:

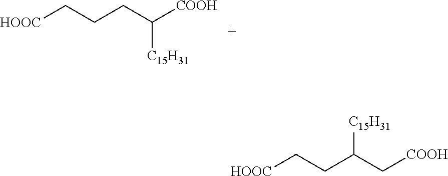

In some embodiments, the compound of formula I is provided as a mixture of diesters:

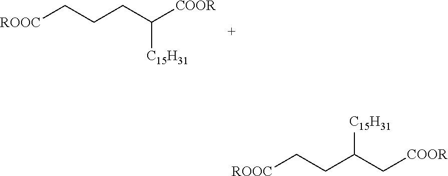

wherein R=alkyl

In some embodiments, the compound of formula I is provided as a mixture of diols:

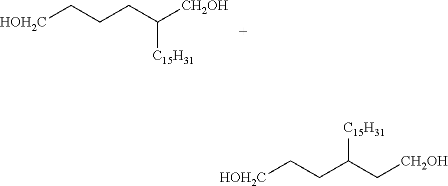

In some embodiments, the compound of formula I is provided as a mixture of diisocyanates:

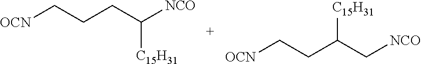

In some embodiments, methods for preparing the compound of formula I are provided. In some embodiments, the methods comprise hydrogenating a cardanol with a hydrogen gas in the presence of at least one catalyst and an optional solvent; maintaining the reaction temperature from about 160° C. to about 180° C. for a period of about 5 hours to about 15 hours and the pressure from about 10 Bar to about 25 Bar; removing the catalyst via filtration to produce a fully hydrogenated product; adding the fully hydrogenated product to a mixture of at least one oxidant and at least one catalyst for a period of about 1 hour to about 3 hours at a temperature below 55° C.; stirring the reaction mixture at temperature from about 60° C. to about 80° C. for a period of about 4 hours to about 20 hours; separating the reaction mixture into an aqueous phase and an organic phase; removing the aqueous phase; adding an air to the organic phase at a temperature from about 80° C. to about 90° C.; washing the organic phase with water; and drying a solid via vacuum to produce the compound.

The hydrogenation of cardanol can be carried out at a temperature from a low of about 30° C. to a high of about 200° C. For example, the temperature can be from about 50° C. to about 190° C., from about 100° C. to about 185° C., or from about 160° C. to about 180° C. In some embodiments, the temperature is from about 160° C. to about 180° C. The hydrogenation of cardanol can be carried out at a pressure from a low of about 5 Bar to a high of about 50 Bar. For example, the pressure can be from about 7 Bar to about 40 Bar, from about 9 Bar to about 30 Bar, or from about 10 Bar to about 25 Bar. In some embodiments, the pressure is from about 10 Bar to about 25 Bar. The length of time for the hydrogenation of cardanol can be from a low of about 5 hours to a high of about 15 hours. For example, the time can be from about 7 hours to about 13 hours, from about 9 hours to about 11 hours, from about 9.5 hours to about 10 hours. In some embodiments, the time is about 10 hours.

The oxidation of 3-pentadecyl-cyclohexanol can be carried out at a temperature from a low of about 50° C. to a high of about 200° C. For example, the temperature can be from about 60° C. to about 160° C., from about 65° C. to about 130° C., or from about 70° C. to about 100° C. In some embodiments, the temperature is from about 70° C. to about 100° C. The number of oxidant moles per each mole of 3-pentadecyl-cyclohexanol is from about 4 to about 20. For example, the moles can be from about 5 to about 15, from about 5.5 to about 12 or from about 6 to about 7. In some embodiments, the moles are from about 6 to about 7.

Illustrative hydrogenation catalysts can include, but are not limited to, Pd/C, Pd(OH)₂, Pd/Al₂O₃, Pd/NaY, Ru-PVP NPs, Ru/C, RuCl₃, Ni and Ni Raney or any combination thereof.

Illustrative oxidation catalysts can include, but are not limited to, ammonium vanadate, copper nitrate, tungstic acid, H₂WO₄ with acidic resins, SBA-15, surfactant-type peroxotungstates, [BMIm]₂WO₄ supported on silica sulphamic acid, H₃PW₁₂O, combinations of Na₂WO₄ with H₂SO₄, Ruthenium- and Cobalt-based sulfophthalocyanines, manganese acetate, cobalt acetate, Co(III)acetylacetonate, Pt/charcoal and tetraalkylammonium halide or any combination thereof.

Illustrative oxidants can include, but are not limited to, hydrogen peroxide, nitric acid, potassium peroxymonosulfate, sodium nitrite/trifluoro acetic acid or any combination thereof.

Illustrative solvents can include, but are not limited to, methanol, ethanol, isopropanol, n-butanol, hexane, cyclohexane, cyclopentane, N-methylpyrrolidone, N,N-dimethylformamide, dimethylsulfoxide, methylene chloride, chloroform, carbon tetrachloride, tetrahydrofuran, water or any combination thereof.

In some embodiments, the cardanol has a purity from about 80% to about 99.5% and preferably from about 95% to about 99.5%.

In some embodiments, the methods comprise reacting a mixture of diacids with a hydroxy compound in the presence of a catalyst; heating the reaction mixture from about 40° C. to about 60° C.; and removing the excess of hydroxy compound by applying vacuum to produce a mixture of diesters as monomers or polymer building blocks. In certain embodiments, the temperature of the reaction between a mixture of diacids and a hydroxy compound is about 20° C., 40° C., 60° C., 80° C., 100° C., 120° C., 140° C., 160° C., 180° C., or 200° C. Any of these values may be used to define a range for the temperature for the reaction between a mixture of diacids and a hydroxy compound. For example, the temperature may range from about 20° C. to about 200° C., from about 30° C. to about 150° C., or from about 40° C. to about 100° C. In some embodiments, the temperature is from about 40° C. to about 60° C. In certain embodiments, the length of time for the reaction between a mixture of diacids and a hydroxy compound is about 1 hour, 3 hours, 5 hours, 8 hours, 10 hours, 13 hours, 15 hours, 18 hours or 20 hours. Any of these values may be used to define a range for the length of time for the reaction between a mixture of diacids and a hydroxy compound. For example, the length of time may range from a low of about 1 hour to a high of about 20 hours, from about 3 hours to about 15 hours, from about 5 hours to about 10 hours. In some embodiments, the time is about 8 hours.

Illustrative catalysts can include, but are not limited to, an acid catalyst, a metal catalyst, or any combination thereof illustrative acid catalysts can include, but are not limited to, sulfuric acid, p-toluenesulfonic acid or any combination thereof illustrative metal catalysts can include, but are not limited to, titanium tetraisopropoxide, dibutyltin (IV) oxide or any combination thereof. In some embodiments, the catalyst is sulfuric acid.

In some embodiments, the methods comprise treating a mixture of diesters with a reducing agent; stirring the reaction mixture at 65° C. for 10 hours; adding an acid to the reaction mixture; extracting the reaction mixture with solvent; washing the organic phase with base and brine; drying the organic phase with metal sulfate; and removing the solvent via vacuum to produce a mixture of diols as monomers or polymer building blocks. In certain embodiments, the temperature of the reaction between a mixture of diesters and a reducing agent is about −25° C., −10° C., 0° C., 30° C., 40° C., 60° C., 80° C., 100° C., 120° C., 140° C., 160° C., 180° C., or 200° C. Any of these values may be used to define a range for the temperature for the reaction between a mixture of diesters and reducing agent compound. For example, the temperature may range from about −25° C. to about 200° C., from about 40° C. to about 150° C., or from about 50° C. to about 100° C. In some embodiments, the temperature is from about 60° C. to about 70° C. In certain embodiments, the length of time for the reaction between a mixture of diesters and a reducing agent is about 5 hours, 8 hours, 10 hours, 15 hours, 20 hours, 25 hours, or 30 hours. Any of these values may be used to define a range for the length of time for the reaction between a mixture of diesters and a reducing agent compound. For example, the length of time may range from a low of about 5 hours to a high of about 30 hours, from about 8 hours to about 20 hours, from about 10 hours to about 15 hours. In some embodiments, the time is about 10 hours.

Illustrative reducing agent can include, but are not limited to, hydrogen, zinc/acetic acid, zinc/hydrochloric acid, lithium aluminum hydride, sodium hydride, sodium cyano-borohydride, sodium borohydride, diisobutyl-aluminium hydride or any combination thereof.

In some embodiments, the methods comprise stirring a mixture of diacids with at least one catalyst; and at least one solvent at 0° C.; converting the mixture of diacids into an acyl azide using an organic azide or an alkyl haloformate and a metal azide at 0° C. or below; stirring the reaction mixture at 0° C. or below for 2 hours; extracting the reaction mixture in solvent; drying the organic phase over metal salt; and removing the solvent under reduced pressure to produce a mixture of diisocyanates as monomers or polymer building blocks. In certain embodiments, the length of time for the reaction between a mixture of diacids and an azide is about 1 hour, 3 hours, 5 hours, 8 hours, 10 hours, 13 hours, 15 hours, 18 hours or 20 hours. Any of these values may be used to define a range for the length of time for the reaction between a mixture of diacids and an azide. For example, the length of time may range from a low of about 1 hour to a high of about 20 hours, from about 3 hours to about 15 hours, from about 5 hours to about 10 hours. In some embodiments, the time is about 2 hours.

Illustrative azides can include, but are not limited to, sodium azide, potassium azide, diphenyl phosphoryl azide or any combination thereof.

Another advantage of the methods and compounds described herein is the validation of isomers, 2-pentadecylhexanedioic acid and 3-pentadecylhexanedioic acid as polymer building blocks, for the synthesis of polyester polymers and polyamide polymers. The isomers, 2-pentadecylhexanedioic acid and 3-pentadecylhexanedioic acid can be used as single source of carboxylic groups in the esterification and amidation reactions. In some embodiments, the cardanol-derivatives can be used from 1% to 100% weight percent of the formulation or in combination with other petro-derivatives or bio-derived raw materials. However, all these raw materials amounts are only exemplary figures and are not intended to be limiting. Other combinations can be used and can be adjusted depending on the specific reagents used.

In some embodiments, a polymer of formula II is provided as:

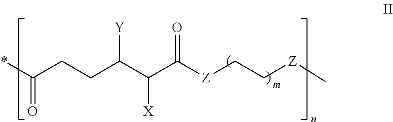

wherein.

X and Y are each independently H and C₁₅H₃₁;

Z is O, NH and S;

m is 1-20; and

n is 1-100.

In some embodiments, the polymer of formula II is provided as a mixture of polyesters:

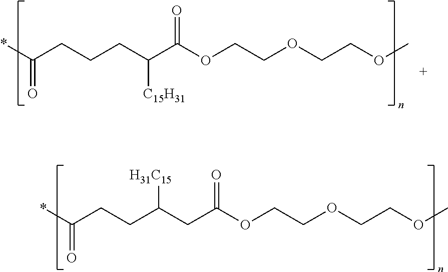

wherein n is 1-50.

In some embodiments, the polymer of formula II is provided as a mixture of polyamides:

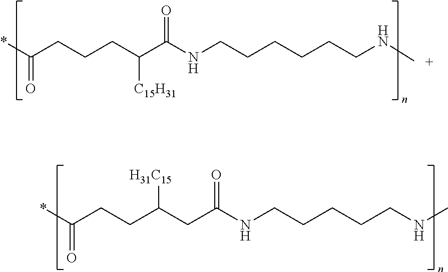

wherein n is 1-50.

In some embodiments, methods for preparing the polymer of formula II are provided. In some embodiments, the methods comprise reacting a mixture of diacids with a diol compound or diol compounds or a hydroxy compound; heating the reaction mixture at a temperature from about 150° C. to about 170° C. for a period of about 1 hours to about 3 hours; adding at least one catalyst to the reaction mixture; maintaining the reaction mixture at a temperature from about 170° C. to about 190° C. for a period of about 10 hours to about 15 hours; and cooling the reaction mixture at a room temperature to produce the polymer. In certain embodiments, the temperature of the reaction between a mixture of diacids and a diol is about 100° C., 110° C., 120° C., 140° C., 160° C., 180° C., 200° C., 220° C., or 250° C. Any of these values may be used to define a range for the temperature for the reaction between a mixture of diacids and a diol compound. For example, the temperature may range from about 100° C. to about 250° C., from about 120° C. to about 220° C., or from about 150° C. to about 200° C. In some embodiments, the temperature is from about 160° C. to about 180° C. In certain embodiments, the length of time for the reaction between a mixture of diacids and a diol is about 2 hour, 3 hours, 5 hours, 8 hours, 10 hours, 13 hours, 15 hours, 18 hours or 20 hours. Any of these values may be used to define a range for the length of time for the reaction between a mixture of diacids and a diol compound. For example, the length of time may range from a low of about 2 hours to a high of about 20 hours, from about 3 hours to about 18 hours, from about 5 hours to about 15 hours. In some embodiments, the time is from about 10 hours to about 14 hours.

Illustrative aromatic diacids can include, but are not limited to, phthalic acid isophthalic acid, terephthalic acid or any combination thereof. Illustrative aliphatic diacids can include, but are not limited to, 1-30 carbons atom dicarboxylic acids, oxalic acid, succinic acid, glutaric acid, adipic acid, pimelic acid, azelaic acid, sebacic acid, citric acid, trimethylolpropionic acid, dimer acids, trimer acids of fatty acid origin or any combination thereof.

Illustrative diols can include, but are not limited to, neopentyl glycol; 2-methyl-1,3-propane-diol; 2-methy-2,4-pentane-diol; 2-butyl-2-ethyl-1,3-propanediol; 2-ethyl-1,3-hexane diol; 2,4-diethyl-1,5-pentane diol; 1,2-propylene glycol; di-propylene glycol; ethylene glycol; diethylene glycol; triethylene glycol; 1,3-propane glycol; butylene glycols; 1,2-cyclohexanediol; polyoxyalkylene polyols; glycerol; 1,1,1-trimethylolpropane; 1,1,1-trimethylolethane; pentaerythritol or any combination thereof.

In some embodiments, the methods comprise reacting a mixture of diacids with a diamine compound or diamine compounds; heating the reaction mixture at a temperature of about 150° C. for a period of about 1 hour; heating the reaction mixture at a temperature of about 180° C. for a period of about 1 hour; and applying a mild vacuum to the reaction mixture at a temperature of about 210° C. for a period of about 1 hour to produce the polymer. In certain embodiments, the temperature of the reaction between a mixture of diacids and a diamine is about 100° C., 110° C., 120° C., 140° C., 160° C., 180° C., 200° C., 220° C., or 250° C. Any of these values may be used to define a range for the temperature for the reaction between a mixture of diacids and a diamine compound. For example, the temperature may range from about 100° C. to about 250° C., from about 120° C. to about 220° C., or from about 150° C. to about 200° C. In some embodiments, the temperature is from about 150° C. to about 180° C. In certain embodiments, the length of time for the reaction between a mixture of diacids and a diamine is about 0.25 hour, 0.5 hour, 1 hour, 2 hours, 3 hours, 5 hours, 8 hours, 10 hours, 13 hours, 15 hours, 18 hours or 20 hours. Any of these values may be used to define a range for the length of time for the reaction between a mixture of diacids and a diamine compound. For example, the length of time may range from a low of about 0.25 hour to a high of about 20 hours, from about 0.5 hour to about 10 hours, from about 1 hour to about 5 hours. In some embodiments, the time is from about 0.5 hour to about 1.5 hours.

Illustrative amines can include, but are not limited to, hydroxylamine; hydroxylamine hydrochloride; diethylenetriamine; tetraethylenepentamine; 1-(1-phenylcyclopentyl)methylamine; 1-hexanamine; ethylenediamine; 2,4-dimethylpentan-3-amine; 2-isopropylaminoethylamine; 2-methylbutan-2-amine; 2N-(3-aminopropyl)-4-aminobutanal; N-isopropyl-2-methylpropan-1,2-diamine; isophoronediamine; sec-butylamine; tert-butylamine; amantadine; butan-1-amine; cyclohexane-1,2-diamine; cyclohexylamine; cyclopropylamine; dicyclohexylamine; ethylamine; isopentylamine; isopropylamine; octadecan-1-amine; octan-1-amine; pentan-1-amine; pentan-3-amine; dimethylamine; diethylmethylamine; 2-aminoethanol; aniline; m-bromoaniline; 2-chloroaniline; 3,5-dichloroaniline; methylamine; 4-methoxyaniline; 3-Nitroaniline; 4-nitroaniline; 4-trifluoromethylaniline; 2,2′-dichloro-4,4′-methylenedianiline (MOCA); 2,4,5-trimethylaniline; 2-methoxyaniline,o-Anisidine; 2-naphthylamine; 3,3′-dichlorobenzidine 3,3′-dichlorobiphenyl-4,4′-ylenediamine; 3,3′-dimethoxybenzidine o-dianisidine; 3,3′-dimethylbenzidine 4,4′-bi-o-toluidine; 4,4′-methylenedi-o-toluidine; 4,4′-oxydianiline; 4,4′-thiodianiline; m-Xylylenediamine; 4,4′-diaminodiphenylmethane (MDA); 4-Aminoazobenzene; 4-chloro-o-toluidine; 4-chloroaniline; 4-methoxy-m-phenylenediamine; -methyl-m-phenylenediamine (toluene-2,4-diamine); 5-nitro-o-toluidine; 6-methoxy-m-toluidine (p-cresidine); benzidine; biphenyl-4-ylamine,4-aminobiphenyl xenylamine; o-aminoazotoluene,4-amino-2′, 3-dimethylazobenzene,4-o-tolylazo-o-toluidine; o-toluidine,2-aminotoluene; tetramethylene diamine; pentamethylene diamine; hexamethylene diamine; decamethylene diamine or any combination thereof.

Other aspects and advantages of these novel cardanol-derived products, as well as their combinations, will be apparent to those skilled in the art. Experimental details are provided in the following examples, which are provided by way of illustration only and should not be construed to limit the disclosure or the appended claims.

### EXAMPLES

In order to provide a better understanding of the foregoing discussion, the following non-limiting examples are offered. Although the examples may be directed to specific embodiments, they are not to be viewed as limiting the invention in any specific respect. All parts, proportions, and percentages are by weight unless otherwise indicated.

### Example 1

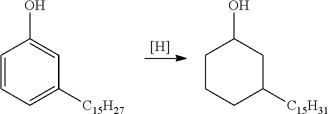

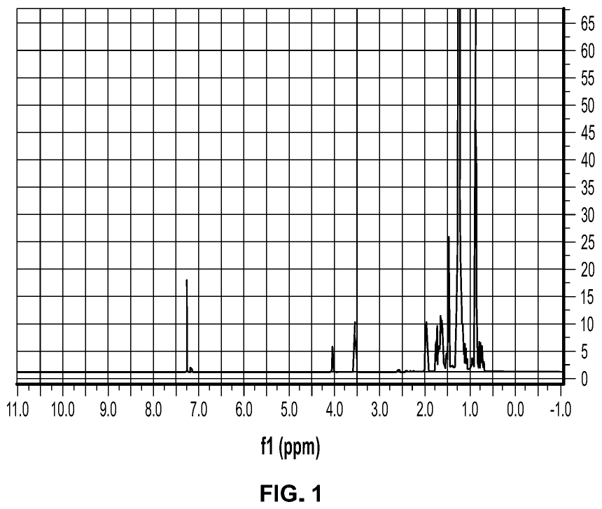

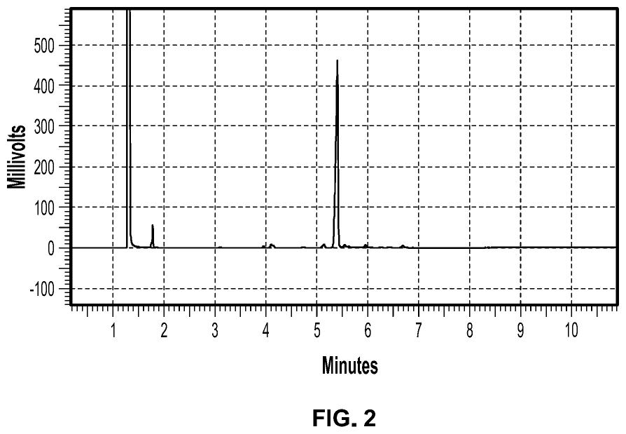

In a Parr reactor, cardanol (300 g; 1 mol) was mixed with Ni catalyst (3% w/w with respect to substrate). The temperature of the reaction mixture was then raised to 170° C. under hydrogen atmosphere (24 Bar) and maintained for 10 hours. The catalyst was removed via filtration over Celite® recovering 3-pentadecyl-cyclohexanol, as a white solid, 85% yield, m.p. 49-51° C. The structure of 3-pentadecyl-cyclohexanol was confirmed and characterized by ¹H-NMR spectrum (FIG. 1) and GC chromatogram (FIG. 2).

### Example 2

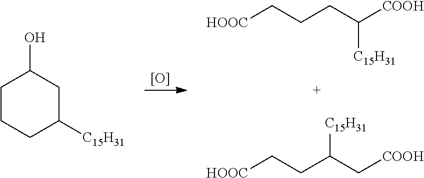

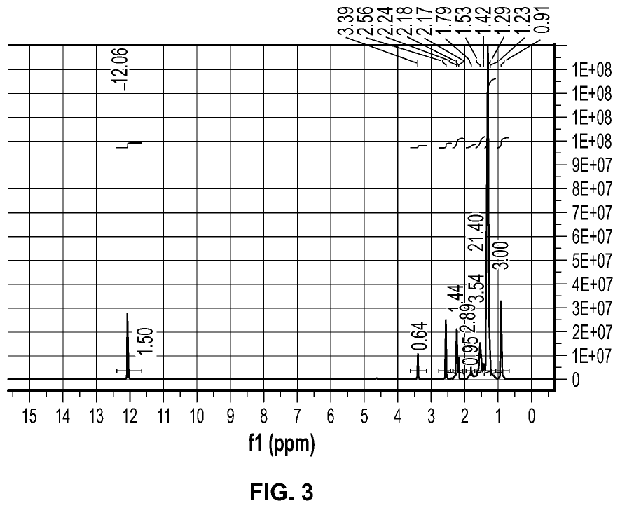

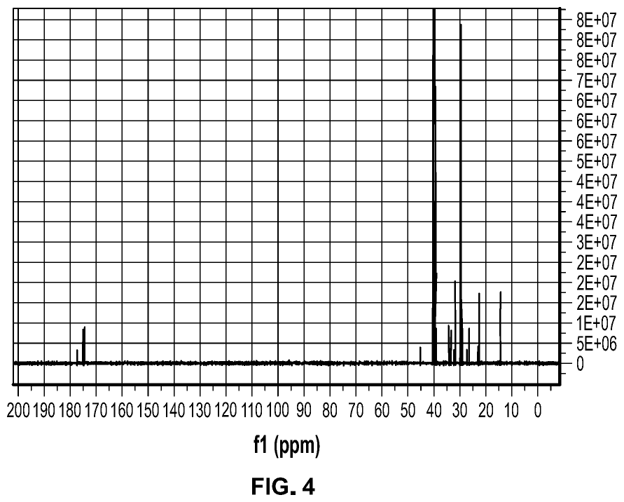

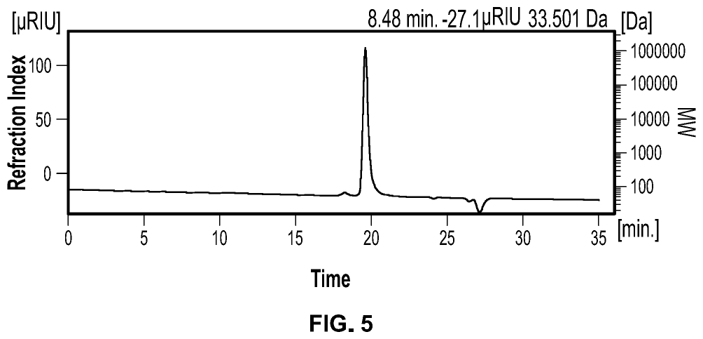

In a reactor equipped with a reflux condenser, inlet for thermocouple, mechanical mixing shaft and inlet for controlled addition of reagents, nitric acid (1080 g, 12.0 mol) (70% solution in water), copper (II) nitrate (4.8 g, 0.02 mol) and ammonium vanadate (3.51 g, 0.03 mol) were added and temperature of the reaction mixture was raised to 50° C. for 30 minutes. 3-Pentadecyl-cyclohexanol (308 g, 1.0 mol) was added to reaction mixture over 2 hours and maintaining the temperature below 55° C. with intermittent external cooling. The reaction mixture was stirred at 70° C. for 4 to 20 hours until reaction is complete. The reaction mixture was separated into two phases, upper organic phase and lower aqueous phase. The lower aqueous phase is discharged. Air was sparged into organic phase at 80-90° C. to destroy remaining nitrogen dioxide. The organic phase was washed twice with water before being vacuum dehydrated and discharged yielding crude white solid, which is a mixture of 2-pentadecylhexanedioic acid and 3-pentadecylhexanedioic acid (m.p. 75° C.). Purity by GC is about 90%. Product can be optionally recrystallized from xylene to yield purified product 2-pentadecylhexanedioic acid and 3-pentadecylhexanedioic acid (m.p. 80° C.) with purity by GC>98%. The structures of 2-pentadecylhexanedioic acid and 3-pentadecylhexanedioic acid were confirmed and characterized by ¹H-NMR spectrum (FIG. 3), ¹³C-NMR spectrum (FIG. 4) and GPC chromatogram (FIG. 5).

### Example 3

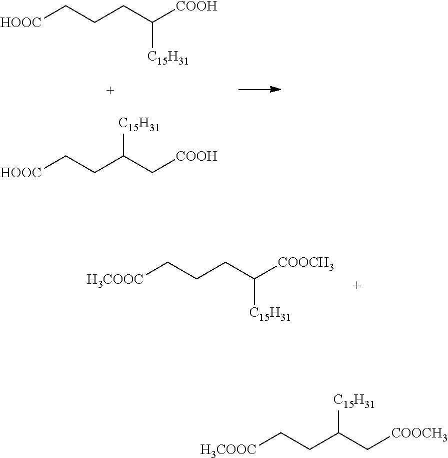

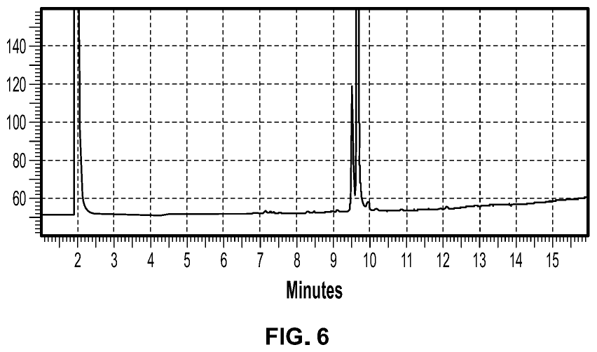

In a glass reactor equipped with Dean-Stark assembly, inlet for thermocouple and an overhead mechanical mixer, 2-pentadecylhexanedioic acid, 3-pentadecylhexanedioic acid (357 g; 1 mol) and methanol (256 g; 8 mol) were added. The catalyst, sulfuric acid (4.9 g; 0.05 mol) was added and the reaction mixture was heated to 50° C. for 8 hours. After the reaction is complete (monitored by acid value titration), vacuum was applied to remove excess alcohol, yielding low viscosity liquid. The structures of methyl 2-pentadecylhexanedioate and methyl 3-pentadecylhexanedioate were confirmed and characterized by GC chromatogram (FIG. 6).

### Example 4

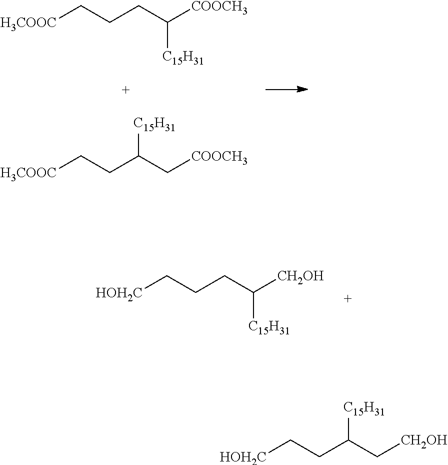

To a mixture of methyl 2-pentadecylhexanedioate and methyl 3-pentadecylhexanedioate (5 mmol) in methanol (30 mL) was slowly added sodium borohydride (15 mmol) under stirring. The reaction mixture stirred at 65° C. for 10 h. Diluted HCl (10%) was then added dropwise to the reaction mixture. The resulting products (pentadecylhexane-1,6-diol as a mixture of isomers) was extracted with diethyl ether (3×30 mL), washed with a 5% NaHCO₃ aqueous solution and brine. The combined organic layers were dried over anhydrous sodium sulfate and the solvent removed under vacuum yielding a mixture of diols.

### Example 5

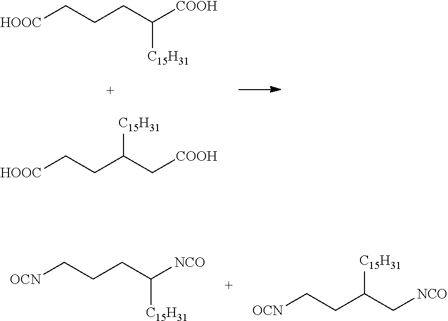

To a mixture of 2-pentadecylhexanedioic acid, 3-pentadecylhexanedioic acid (10 g, 0.028 mol) and triethylamine (7.8 ml, 0.056 mol) was added tetrahydrofuran (15 ml) under stirring at 0° C. Ethyl chloroformate (5.9 mL, 0.0616 mol) was added dropwise at 0° C. The reaction mixture was stirred at 0° C. for 2 h followed by dropwise addition of a solution of sodium azide (29.1 g, 0.448 mol) in water (35 mL) at 0° C., maintaining the reaction mixture at 0° C. for 2 h. The reaction mixture was extracted with diethyl ether (3×50 ml). The combined organic layers were dried over anhydrous sodium sulfate and the solvent removed under reduced pressure yielding a mixture of diisocyanates.

### Example 6

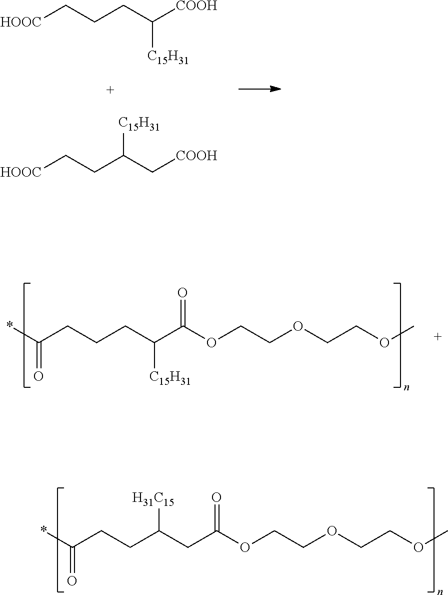

In a glass reactor equipped with Dean-stark assembly and an overhead condenser, 2-pentadecylhexanedioic acid, 3-pentadecylhexanedioic acid (357 g; 1 mol) and ethylene glycol (74.4 g; 1.2 mol) were added. The reaction mixture was heated at 160° C. for 2 hours followed by an addition of a catalyst, di-n-butyltin oxide (0.8 g; 0.004 mol) and maintaining the reaction temperature at 180° C. for 10-14 hours. The reaction temperature was cooled to room temperature yielding viscous polyester polymer.

### Example 7

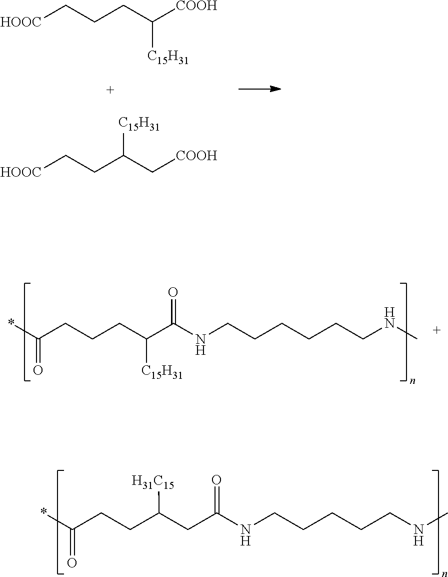

In a reactor equipped with reflux condenser, mechanical mixer and an inlet for thermocouple, 2-pentadecylhexanedioic acid, 3-pentadecylhexanedioic acid (357 g, 1.0 mol) and hexamethylenediamine (116.2 g; 1.0 mol) were added. The reaction mixture was heated at 150° C. for 1 hour followed by further heating at 180° C. for 1 hour. Mild vacuum was applied to the reaction mixture at a temperature of about 210° C. for a period of about 1 hour yielding polyamide polymer.

### INDUSTRIAL APPLICABILITY

The major advantage of the present invention is that the use of cardanol-derived regio-isomers, 2-pentadecylhexanedioic acid and 3-pentadecylhexanedioic acid as a bio-derived alternative to the well-known adipic acid, as well as other dicarboxylic acids. In fact, 2-pentadecylhexanedioic acid and 3-pentadecylhexanedioic acid can be used in the production of polyester diols, polyols, polyamides (e.g. bio-analogs of Nylon 6 or polyamide Nylon 6,6), plasticizers and lubricants. These diacids, 2-pentadecylhexanedioic acid and 3-pentadecylhexanedioic acid impart flexibility even at low temperature, amorphous properties, improved chemical resistance properties and hydrophobicity due to the presence of the —C₁₅H₃₁ group, in the final thermoset and thermoplastic polymer matrices.

Cardanol is one of the most promising bio-based material used in the thermoset-industry, by derivatizing through the aromatic ring, the phenolic OH or the side chain double bonds. All the resulting products offer unique features like thermal resistance, high hydrophobicity and chemical resistance, flexibilization effect if the side chain is not modified, low volatility (therefore helping with VOC reduction when low viscosity cardanol-derivative are used as replacement to potentially dangerous organic solvents).

The present invention provides vital choice of high purity cardanol as starting substrate, its conversion to fully hydrogenated cardanol and subsequent derivatization under proper conditions leading to the development of a series of novel cardanol-derivatives. These structures can overcome some of the well-known limitations of cardanol (e.g. UV instability, batch-to-batch consistency) as well as offering novel chemical tools to impart unique chemical and mechanical properties (e.g. low temperature flexibility) to both thermosetting and thermoplastic matrices, extending the only nowadays-limited applicability of cardanol and its derivatives in thermoplastics.

The compounds and methods of making the same provided for in the present application can be used in many methods/applications. Examples include, but are not limited to, the use as raw materials for coatings, linings, adhesives, alkyds, varnishes, composites, inks, structural polymers, 3D printable polymers, techno-polymers and elastomers.

In some embodiments, methods for using a compound of formula I and a polymer of formula II in antimicrobials, antioxidants, adhesives, coatings, corrosion retardants composites, cosmetics, detergents, soaps, de-icing products, elastomers, food, flavors, inks, lubricants, oil field chemicals, personal care products, polymers, structural polymers, engineered plastics, 3D printable polymers, techno-polymers, rubbers, sealants, solvents, surfactants, varnishes etc. are provided.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges appear in one or more claims below. All numerical values are “about” or “approximately” the indicated value, and take into account experimental error and variations that would be expected by a person having ordinary skill in the art.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

What is claimed is:

1. A compound of formula I: 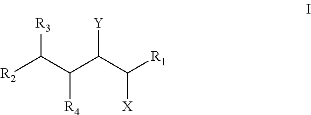
wherein:
X and Y are each independently H and C₁₅H₃₁;
R₁ and R₂ are each independently —COOH, —COOR₅, —CO-halogen, —CH₂OH, —CH₂OR₅, —COSR₅, —CONH₂, —CH₂NH₂, CH₂NHR₅, —CH₂N₃R₅, —NCO, —CH₂-halogen, —CH₂OCH₂(CHCH₂O), —CHO, —CN, —CONH—CO—R₅ and —CH₂O—CO—O—R₅; and
wherein R₅ is H, alkyl, alkenyl, alkoxy, cycloalkyl, aryl, heteroaryl, and —CH₂—N—(CH₂CH—R₆—O)₂—(CH₂CH—R₆—O)₀₋₁₀—H; and
wherein R₆ is H or alkyl;
R₃ and R₄ are each independently H, alkyl, alkoxy and cycloalkyl.

2. The compound of claim 1, wherein the compound of formula I is a mixture of diacids:

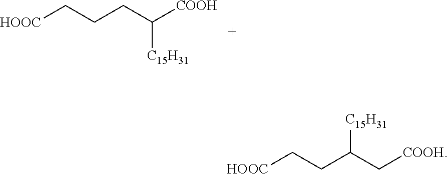

3. The compound of claim 1, wherein the compound of formula I is a mixture of diesters: 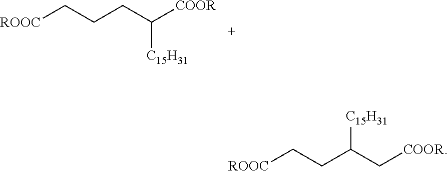
wherein R=alkyl

4. The compound of claim 1, wherein the compound of formula I is a mixture of diols:

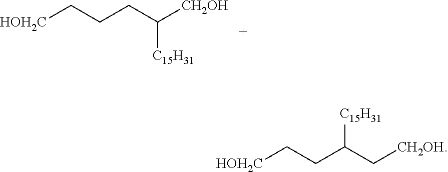

5. The compound of claim 1, wherein the compound of formula I is a mixture of diisocyanates:

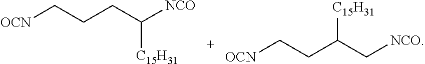

6. A method for preparing a compound of claim 1, comprising:
hydrogenating a cardanol with a hydrogen gas in the presence of at least one catalyst; and an optional solvent as reactants;
heating the reactants at a predetermined temperature and at a predetermined pressure for a predetermined period of time;
removing the catalyst via filtration to produce a fully hydrogenated product;
adding the fully hydrogenated product to a mixture of at least one oxidant and at least one catalyst for a predetermined period of time at a temperature below 55° C.;
stirring the reaction mixture at a predetermined temperature for a predetermined period of time;
separating the reaction mixture into an aqueous phase and an organic phase;
removing the aqueous phase;
adding an air to the organic phase at a predetermined temperature;
washing the organic phase with water; and
drying a solid via vacuum to produce the compound.

7. The method of claim 6, wherein the hydrogenation catalyst comprises Pd/C, Pd(OH)₂, Pd/Al₂O₃, Pd/NaY, Ru-PVP NPs, Ru/C, RuCl₃, Ni, and Ni Raney or any combination thereof; and
wherein the oxidation catalyst comprises ammonium vanadate, copper nitrate, tungstic acid, H₂WO₄ with acidic resins, SBA-15, surfactant-type peroxotungstates, [BMIm]₂WO₄ supported on silica sulphamic acid, H₃PW₁₂O, combinations of Na₂WO₄ with H₂SO₄, Ruthenium- and Cobalt-based sulfophthalocyanines, manganese acetate, cobalt acetate, Co(III)acetylacetonate, Pt/charcoal and tetraalkylammonium halide or any combination thereof.

8. The method of claim 6, wherein the oxidant comprises hydrogen peroxide, nitric acid, potassium peroxymonosulfate, sodium nitrite/trifluoro acetic acid or any combination thereof.

9. The method of claim 6, wherein the cardanol has a purity from about 95% to about 99.5%.

10. The method of claim 6, further comprising:
reacting a mixture of diacids with a hydroxy compound in the presence of a catalyst;
heating the reaction mixture at a predetermined temperature for a predetermined period of time; and
removing the excess of hydroxy compound by applying vacuum to produce a mixture of diesters.

11. The method of claim 10, wherein the catalyst comprises an acid catalyst, a metal catalyst or any combination thereof.

12. The method of claim 10, further comprising:
treating a mixture of diesters with a reducing agent;
stirring the reaction mixture at a predetermined temperature for a predetermined period of time;
adding an acid to the reaction mixture;
extracting the reaction mixture with a solvent;
washing the organic phase with base and brine;
drying the organic phase with metal sulfate; and
removing the solvent via vacuum to produce a mixture of diols.

13. The method of claim 12, wherein the reducing agent comprises hydrogen, zinc/acetic acid, zinc/hydrochloric acid, lithium aluminum hydride, sodium hydride, sodium cyano-borohydride, sodium borohydride, diisobutyl-aluminium hydride or any combination thereof.

14. The method of claim 6, further comprising:
stirring a mixture of diacids with at least one catalyst; and at least one solvent at 0° C.;
converting the mixture of diacids into an acyl azide using an organic azide or an alkyl haloformate and a metal azide at 0° C. or below;
stirring the reaction mixture at 0° C. or below for a predetermined period of time;
extracting the reaction mixture in a solvent;
drying the organic phase over metal salt; and
removing the solvent under reduced pressure to produce a mixture of diisocyanates.

15. The method of claim 14, wherein the azide comprises sodium azide, potassium azide, diphenyl phosphoryl azide or any combination thereof.

16. A polymer of formula II: 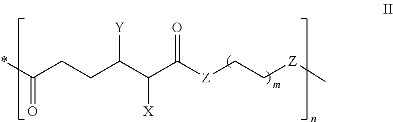
wherein:
X and Y are each independently H and C₁₅H₃₁;
Z is O, NH and S;
m is 1-20; and
n is 1-100.

17. The polymer of claim 16, wherein the polymer of formula II is a mixture of polyesters: 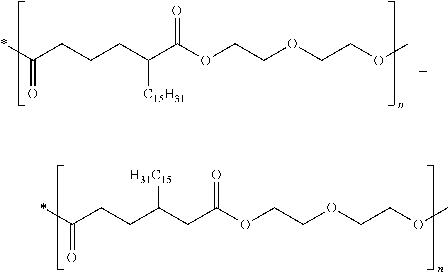
wherein n is 1-50.

18. The polymer of claim 16, wherein the polymer of formula II is a mixture of polyamides: 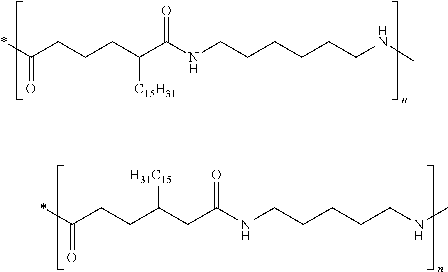
wherein n is 1-50.

19. A method for preparing a polymer of claim 16, comprising:
reacting a mixture of diacids with a diol compound or diol compounds or a hydroxy compound;
heating the reaction mixture at a predetermined temperature for a predetermined period of time;
adding at least one catalyst to the reaction mixture;
maintaining the reaction mixture at a predetermined temperature for a predetermined period of time; and
cooling the reaction mixture at a room temperature to produce the polymer.

20. A method for preparing a polymer of claim 16, comprising:
reacting a mixture of diacids with a diamine compound or diamine compounds;
heating the reaction mixture at a predetermined temperature for a predetermined period of time;
heating the reaction mixture at a predetermined temperature for a predetermined period of time; and
applying a mild vacuum to the reaction mixture at a predetermined temperature for a predetermined period of time to produce the polymer.
